(19) United States
(12) Patent Application Publication
(10) Pub. No.: US 2009/0117131 A1
(43) Pub. Date: May 7, 2009

(54) Pharmaceutical Composition for The Treatment or Prevention of Allergic Diseases, Use Thereof, and A Method for The Treatment or Prevention of Allergic Diseases

(76) Inventors: Sook-Yeong Jeon, Gyeonggi-do (KR); Dong Ho Nahm, Gyeonggi-do (KR)

Correspondence Address: THE RAFFERTY PATENT LAW FIRM, 1952 Gallows Road, Suite 200, Vienna, VA, 22182-3823, US

(21) Appl. No.: 12/294,042

(22) PCT Filed: Mar. 20, 2007
(86) PCT No.: PCT/KR07/01350

§ 371 Date: Sep. 22, 2008

(30) Foreign Application Priority Data
Mar. 20, 2006 (KR) 10-2006-0025175
Sep. 15, 2006 (KR) 10-2006-0089746
Mar. 19, 2007 (KR) 10-2007-0026845

(51) Int. Cl. A61K 39/395; A61P 37/08; A61P 11/06; A61P 19/10; A61P 37/00

(52) U.S. Cl. 424/171.1

(57) ABSTRACT

The present invention provides a pharmaceutical composition for preventing or treating allergic diseases comprising histamine and allergen-specific antibody as active ingredients. Also, the present invention provides a use of the above composition for the manufacture of medicament for preventing or treating allergic diseases. Further, the present invention provides a method of preventing or treating allergic diseases which comprises administrating the above pharmaceutical composition to a mammal. When the pharmaceutical composition of the present invention is administered to a mammal, a specific allergic immune response (hypersensitive immune response) to antigen inducing allergic reaction (allergen) that can not be effectively controlled by current standard pharmacological treatments can be effectively suppressed. Therefore, refractory allergic diseases that cannot be sufficiently improved by the current standard pharmacological treatments can be effectively improved if the pharmaceutical composition of the present invention, its use for preventing or treating allergic diseases, or a method of preventing or treating allergic diseases by the above composition is applied.

### TECHNICAL FIELD

The present invention provides a pharmaceutical composition for preventing or treating allergic diseases comprising histamine and allergen-specific antibody as active ingredients. The present invention also provides a use of the above pharmaceutical composition for the manufacture of a medicament for preventing or treating allergic diseases. Further, the present invention relates a method of preventing or treating an allergic diseases comprising administering the above pharmaceutical composition to a mammal.

### BACKGROUND ART

Historically, the allergy has been defined as a phenomenon that hypersensitive response to a certain antigenic material inducing harmful damage to the host who developed the hypersensitive response (Aronson J. BMJ 1999;319:308). Accordingly, the allergy is an opposite term to immunity that is defined as a response to a certain antigenic material inducing beneficial response to the host who developed this response (Aronson J. BMJ 1999;319:308). Recently, allergic reaction frequently indicates IgE antibody-mediated immediate type hypersensitivity reaction (hypersensitivity reaction type I) to external environmental antigens in narrow meaning of allergic reaction. However, allergic reaction in wide meaning indicates all kinds of hypersensitive reaction to various antigens existing in the external environment or inside of the host mammal. Accordingly, hypersensitive response to self antigenic protein (autoantigen) has been termed as autoallergy (Aronson J. BMJ 1999;319:308). Accordingly, allergic reaction in wide meaning indicates all kinds of hypersensitive immunological reactions (that is classified as type I, type II, type III, and type IV hypersensitivity reaction according to Gell and Coombs' classification of hypersensitivity reaction) and their resulting phenomena that can be harmful to the host (Bierman C W, et al. (eds.) Allergy, asthma, and immunology from infancy to adulthood. page xvii, Saunders, Philadelphia, 1996).

Allergic diseases are known to be developed from damage of organ in the body due to allergic reaction. And the allergic diseases include bronchial asthma, atopic dermatitis, allergic rhinitis, allergic conjunctivitis, and urticaria (Bierman C W, et al. (eds.) Allergy, asthma, and immunology from infancy to adulthood. page xvii, Saunders, Philadelphia, 1996). And allergic diseases also include rheumatoid arthritis and systemic lupus erythematosus which is known to be developed from hypersensitive reaction (including autoantibody response) to self antigen (autoantigens including immunoglobulin, collagen, DNA) and has been classified as autoallergic diseases (Vaughan J H. Med Times 1969;97:187-204). Recently, rheumatoid arthritis, systemic lupus erythematosus, and pemphigus which had been historically classified as autoallergic diseases became to be called as “autoimmune diseases” (Aronson J. BMJ 1999;319:308; Davidson, A. et al., N Engl J Med 2001; 345:340-350). The term of autoimmune disease is misnomer and there has been several critics on the use of this terminology but this term become a generalized habitual term in the related academic society and industrial fields. Some researchers suggested a new term called “autoaggressive diseases” instead of autoimmune diseases, but this terminology has not been widely used also.

Generally, an antigenic material that can induce allergic reaction is defined as an allergen in the related academic and industrial fields. So, the allergen includes common environmental antigens that are known to frequently induce allergic reaction such as house dust mite, pollen, animal dander, fungus, egg, ovualbumin, milk protein, wheat flour, peanut, etc.

Besides, the above allergic diseases including atopic dermatitis, bronchial asthma, allergic conjunctivitis, allergic rhinitis, and urticaria have been known to be developed by similar pathogenetic mechanisms although patterns of their clinical manifestations are different. Accordingly, there are many patients who are suffering from more than 2 kinds of above allergic diseases at the same time.

A specific IgE antibody response to allergen plays a central role in the development of allergic diseases regardless the kinds of causative external allergens such as inhalant allergens including house dust mite and pollens or food allergens including egg, ovualbumin, milk protein, wheat flour and peanut. The critical central role of allergen-specific IgE antibody response in the development of allergic diseases can be confirmed by recent reports showing that suppression of IgE-mediated allergic reaction with administrations of humanized monoclonal anti-IgE antibody can significantly improve the clinical symptoms in the patients having bronchial asthma, allergic rhinitis, allergic conjunctivitis, food allergy, and atopic dermatitis (Chang T W, et al. J Allergy Clin Immunol 2006;117:1203-12). Furthermore, allergic response to specific allergen induced in a mammal and physiological changes resulting from allergic response to specific allergen in the mammal can be reproduced in the other mammal by passive transfer of allergen-specific IgE antibody to the other mammal regardless the kinds of allergens (food allergens like ovualbumin or inhalant allergens like house dust mite) (Oshiba A, et al. J Clin Invest 1996;97:1398-408; Herz U, et al. Clin Exp Allergy 2004;34:478-87). The above experiments on the passive transfer of allergic reaction by allergen-specific IgE antibody prove that allergen-specific IgE antibody plays a critical common central role not only in the allergic reaction but also in the developments of various allergic diseases and the allergen-specific IgE antibody is a critical therapeutic target. Allergen-specific IgE antibody is confirmed to play the most important central role in the development of allergic diseases induced by allergic reaction to external antigens on the basis of comprehensive analysis of past research results (Platts-Mills T A. Am J Respir Crit Care Med 2001;164:S1-5).

Currently, three kinds of treatment methods are used for the treatment of allergic diseases caused by external environmental allergen and these methods include avoidance of causative allergen, allergen-immunotherapy, and the use of drugs. Of these, avoidance of the allergen is a method to avoid exposure to causative allergens identified by allergy skin test. Of these, pharmaceutical treatment is administrations of drugs (corticosteroids, antihistamine, and leukotriene antagonists, etc.) that can suppress an allergic reaction and inflammation of various tissues induced by allergic reaction. Of these, allergen-immunotherapy is a treatment that causative allergen is administered to the patients with allergic diseases from the small dose to increasing dose through a subcutaneous route and thereby induces selective decrease of an allergic reaction to the administered allergen. Although the above treatment methods can improve the clinical symptoms in many patients with allergic diseases, there are still significant numbers of patients that can not experience sufficient clinical improvements and can not be satisfied by the above treatments. Especially, an effective avoidance of environmental allergen is practically very difficult in a real daily living. There have been reports about cases of patients who were died due to severe allergic reactions after allergen-immunotherapy. The practice rate of allergen-immunotherapy in the management of patients with allergic diseases is known to be widely different according to the countries due to the safety issues including a possible development of acute severe allergic reaction that can cause death (anaphylaxis). Currently, there are urgent needs for the developments of a new treatment method that can fundamentally improve the allergic diseases due to environmental antigens by selectively decreasing a hypersensitivity reaction to specific antigen and that is easy to practice and have no side effects. Recently, the incidences of the allergic diseases are known to rapidly increase world-widely. However a development of an effective preventive method has not been possible yet.

On the other hand, many chronic inflammatory diseases including systemic lupus erythematosus, rheumatoid arthritis, pemphigus, and dilated cardiomyopathy are known to be autoallergic diseases that are developed by a hypersensitivity reaction to self proteins (autoallergic reaction) and subsequently develop specific organ damage by the specific IgG antibody to autoallergen (self-antigen) (Vaughan J H. Med Times 1969;97:187-204; Albin R J. Lancet 1968;2(7583):1397; Davidson, A. et al., N Engl J Med 2001; 345:340-350).

Currently, the major treatment of autoallergic diseases are treatment with corticosteroids or immunosuppressants that decrease the inflammation or suppress the immunity nonspecifically regardless the kinds of causative autoallergens. However, there are still significant numbers of patients that can not experience sufficient clinical improvements even after the above pharmacological treatments. The patients with recalcitrant autoallergic diseases that can not be controlled by the standard pharmacological therapy were shown to be clinically effective by following three treatment methods according to the past reports. Firstly, plasma exchange therapy removes the plasma of patients and administers plasma from normal subject. Secondly, selective apheresis removes the IgG antibody in the plasma externally drawn from the patients by passing a plasma of the patients through Protein-A column and the remaining components of plasma are administered to the same patients (Bosch T. Ther Apher Dial 2005;9:459-68). Thirdly, antigen-specific antibody apheresis removes autoallergen-specific autoantibody in the plasma from the patients by autoallergenic protein and returning the other remaining components of plasma to the same patients (Schimke I, et al. J Clin Aphersis 2005;20:137-142). Therefore, it is evident that specific antibody to autoallergenic protein plays a most critical central role in the development of autoallergic diseases. However, the above plasmapheresis or selective apheresis treatment method nonspecifically removes total IgG antibody from plasma and recently developed antibody treatment that induce depletion of immunoglobulin producing B lymphocyte also results in reduction of total immunoglobulin amounts. Therefore, the above nonspecific immunoglobulin reduction methods do not selectively decrease specific immune response to causative allergens and have a significant risk for the development of fatal side effects including decreased resistance to infection (Edwards J C, et al. Rheumatology 2005;44:151-6). And the above method that removes autoallergen-specific autoantibody from the plasma drawn externally from the patients by autoallergenic protein and returning the other remaining components of plasma to the same patients is very complex procedure and has a risk of developing hypotension during the procedure. Accordingly, there are urgent needs for the developments of new effective treatment methods that can fundamentally improve the allergic diseases on the basis of pathogenetic mechanism and that are also safe and easy to practice.

If a certain kind of specific antibody is injected to animal, an antibody (called as anti-idiotype antibody) to the antigen-binding portion (idiotype) of the initially administered specific antibody is produced in the animal and this anti-idiotype antibody can decrease the amounts of the initially administered specific antibody in the animal (Shoenfeld Y, et al. Int Arch Allergy Immunol 1994;105:211-23). Anti-idiotype antibody is also known to regulate IgE antibody response in human (Geha, et al. J Clin Invest, 1983). The self regulating mechanism suppressing the production of a specific antibody is present in normal healthy person by the development of anti-idiotype antibody in case of overproduction of the certain from of antibody (Zouali M, et al. Autoimmunity 1996;24:55-63). Accordingly, an administration of antigen-specific antibody purified from human or other mammals with allergic diseases to human with allergic diseases is likely to decrease the allergen-specific antibody or autoallergen (or autoantigen)-specific antibody and thereby clinically improve the patients with allergic diseases including autoallergic diseases that an overproduction of antibody to specific allergen plays a central role in the development of disease (Geczy A F, et al. 1978;62:261-70; Zouali M, et al. Autoimmunity 1996;24:55-63). However, the clinical efficacy of the above treatment inducing anti-idiotype antibody has not been proven in the human allergic diseases and administration of allergen-specific antibody alone has failed to produce clinical improvement of allergic diseases in one past report (Saint-Remy J M. Ad Exp Med Biol 1996;409:417-24). Currently, the above immunotherapy using allergen-specific antibody is not applied in the real clinical practice for treating allergic diseases, yet. Accordingly, there are urgent needs of developing a new advanced effective pharmaceutical composition for immunotherapy to induce anti-idiotype antibody response advanced from the simple administration of allergen-specific antibody alone.

In 1951, Parrot and Laborde developed a new treatment method administering a complex of histamine and human serum gammaglobulin to restore the ability of blocking a biologic activity of histamine (histaminopexy) that is decreased in the sera of patients with allergic diseases (J Physiol 1951;40:885-9). A complex of histamine/human gammaglobulin has been widely used in many countries including Japan and Europe for the treatment of allergic diseases including allergic rhinitis, bronchial asthma, atopic dermatitis, and chronic urticaria (Yoshii H, et al. J Allergy Clin Immunol 1997;100:809-16). The treatment method using histamine-immunoglobulin complex is sometimes called as ‘nonspecific immunotherapy.’ Injection of histamine-immunoglobulin complex to animal model of allergy induced reductions of allergic inflammatory response and also showed an immunomodulating effects inducing reductions of the serum levels of TNF-alpha, and IL-4 (Ayoub M, et al. Int Immunopharmacol 2003;3:523-539). The histamine-immunoglobulin complex has been also reported to show an anti-inflammatory effect in an animal model of autoallergic disease (U.S. Pat. No. 6,627,194). The anti-inflammatory effect of a complex of histamine-immunoglobulin (or gammaglobulin) was not produced when the same amount of histamine or immunoglobulin alone or histamine-albumin complex or serotonin-immunoglobulin complex was administered to the animal model of allergy (Yoshii H, et al. J Allergy Clin Immunol 1997;100:809-16). Accordingly, a specific interaction between histamine and immunoglobulin and a combination of both histamine and immunoglobulin were supposed to have an important role for the pharmacological effects of this histamine-immunoglobulin complex for the treatment of allergic diseases. However, this “nonspecific immunotherapy” using histamine-immunoglobulin complex is not recommended as a standard pharmacological treatment in the international guidelines for treatment of allergic diseases including atopic dermatitis and bronchial asthma and in the textbooks of allergic diseases (Hanifin J M, et al. J Am Acad Dermatol 2004;50:391-404; Global Initiative for Asthma: NIH publication no. 02-3659, 2002; Bierman C W, et al. (eds.) Allergy, asthma, and immunology from infancy to adulthood. page xvii, Saunders, Philadelphia, 1996). And the clinical effectiveness of histamine-immunoglobulin complex is not significantly better than the effectiveness of other current standard pharmacological treatments and the proportion of patients showing significant therapeutic efficacy by the treatments with histamine-immunoglobulin complex alone is not significantly higher than the other above treatments (Kukita J. Nishinipponhifuka 1980;42:470-7). Moreover, the exact pharmaceutical action mechanism of currently used histamine-immunoglobulin complex producing clinical therapeutic effects in the patients with allergic diseases including bronchial asthma, chronic rhinitis, allergic conjunctivitis, atopic dermatitis, urticaria, rheumatoid arthritis has not been clearly defined yet.

Immunoglobulin contained in the histamine-immunoglobulin complex currently used for the treatment of allergic diseases is purified from a mixture of plasma materials (pooled plasma) donated by multiple healthy volunteers (usually from more than 1000 volunteers). In the process of manufacturing a immunoglobulin contained in the histamine-immunoglobulin complex, plasma materials showing negative test for the presence of infectious virus were selected and mixed to make a pooled plasma and the immunoglobulin is purified from the above pooled plasma as the current manufacturing processes of various other current immunoglobulin preparations for injection use (Martin T D. Int Immunopharmacol 2006;6:517-22). The present inventors discovered that tests evaluating the presence or the titer of specific antibody to a specific antigen causing allergic reaction (allergen) are not included in the current screening process for selecting plasma materials that will be used for the purification of immunoglobulin to manufacture a histamine-immunoglobulin complex except for the tests for the antibody to infectious virus or infectious bacteria. Accordingly, the present inventors judged that a new advanced pharmaceutical composition like a pharmaceutical composition of the present invention is needed. The present inventors judged that an uncertainty including unpredictability and incompleteness of therapeutic efficacy of the currently used histamine-immunoglobulin complex in the treatment of human allergic diseases is originated from uncertainty of antigen-specificity of immunoglobulin used for the manufacture of the histamine-immunoglobulin complex. And there has been no trial to manufacture a new pharmaceutical composition of histamine-immunoglobulin complex for treating allergic diseases using immunoglobulin purified from plasma materials selected by the tests for the presence or the titer of specific antibody to allergen. Accordingly, the present inventors judged that a noble advanced pharmaceutical composition for preventing and treating allergic diseases can be created by a specific combination of histamine and the immunoglobulin that can specifically bind to specific allergen (allergen-specific antibody). And the present inventors judged that the above pharmaceutical composition of the present invention can have not only anti-allergic and anti-inflammatory effects by the combination of histamine and immunoglobulin of uncertain antigen-specificity as reported in the previous experiments using animal model but also produce an anti-allergic effect by administration of allergen-specific antibody to animal for inducing an production of anti-idiotype antibody as reported in the previous animal model experiments and thereby decreasing the amounts of allergen-specific antibody.

Especially, the present inventors created an idea to develop a pharmaceutical composition for preventing and treating allergic diseases comprising histamine and allergen-specific hyperimmune globulin containing allergen-specific antibody on the basis of the current practices and proven clinical efficacies of virus antigen-specific hyperimmune globulin in the prevention of hepatitis B virus and cytomegalovirus (Snydman D R. Transpl Infect Dis 2001;3(suppl 2):6-13; Gelfand E W. J Allergy Clin Immunol 2001;108(4 suppl):S111-6). The virus antigen-specific hyperimmune globulin (ex, cytomegalovirus hyperimmune globulin) is currently manufactured by using immunoglobulin made from selected plasma materials either naturally having high titers of specific antibody to the above viruses among the plasma materials from blood donations or made from plasma materials of volunteers having high titers of specific antibody to the above viruses by intended immunizations to the above viruses.

### DISCLOSURE OF INVENTION

### Technical Problem

The present inventors made great efforts to develop a more effective pharmaceutical composition and a new treatment method for patients with allergic diseases that are not effectively controlled by current standard pharmacological therapies. As one of these efforts, the present inventors hypothesized that a noble advanced pharmaceutical composition for preventing and treating allergic diseases can be created from a specific combination of histamine and allergen-specific antibody manufactured from immunoglobulin purified from selected blood materials of mammal containing high concentration of specific antibody to a certain allergen can show an significantly excellent anti-allergic effect suppressing the production of allergen-specific antibody compared to the currently used histamine-immunoglobulin complex. The present inventors demonstrated the above hypothesis by the examples of the present invention and thereby completed the present invention. And the present inventors also tried to develop a preventive vaccine for allergic diseases by administering a pharmaceutical composition of the present invention comprising histamine and allergen-specific antibody as active ingredients to the mammal with high risk for developing allergic diseases before the development of allergic diseases.

### Technical Solution

The present invention provides a pharmaceutical composition for preventing or treating allergic diseases comprising histamine and allergen-specific antibody as active ingredients.

In an embodiment of the present invention, the allergen-specific antibody can be an immunoglobulin capable of specifically reacting with allergen.

In an embodiment of the present invention, the allergen-specific antibody can be an allergen-specific hyperimmune globulin.

In an embodiment of the present invention, the allergen-specific antibody can be an allergen-specific IgG antibody.

In an embodiment of the present invention, the preventing or treating allergic diseases can be resulted from the suppression of allergen-specific IgE antibody response. Also, the preventing or treating allergic diseases can be additionally resulted from the suppression of allergen-specific IgG antibody response.

In an embodiment of the present invention, the allergen can be one or more of allergen(s) selected from a group consisting of egg, ovualbumin, milk, shrimp, crab, wheat, peanut, house dust mite, pollen, animal dander and fungus.

Also, in an embodiment of the present invention, the allergen can be one or more of allergen(s) selected from a group consisting of nuclear antigen protein, double stranded DNA, phospholipid, beta-2 glycoprotein I, Fc fragment of human IgG antibody and type II collagen.

In an embodiment of the present invention, the above allergic diseases can be atopic dermatitis, allergic rhinitis, allergic conjunctivitis, urticaria or bronchial asthma.

Also, in an embodiment of the present invention, the allergic diseases can be systemic lupus erythematosus or rheumatoid arthritis.

The present invention provides a method of manufacturing a pharmaceutical composition for preventing or treating allergic diseases, which comprises the following steps of: (a) immunizing a mammal with an allergen, (b) measuring titers of allergen-specific immunoglobulin that can react with a certain allergen in the blood samples from the mammal, (c) screening and selecting a mammal plasma having higher titers of allergen-specific immunoglobulin in the plasma with at least 2 fold higher serum dilution titer compared to normal mammals of same species, (d) purifying allergen-specific immunoglobulin from the mammal plasma containing high titers of allergen-specific immunoglobulin selected from the above step (c), (e) mixing the allergen-specific immunoglobulin purified in the above step (d) with histamine.

The present invention also provides a method of manufacturing a pharmaceutical composition for preventing or treating allergic diseases, which comprises the following steps of: (a) measuring titers of allergen-specific immunoglobulin that can react with a certain allergen in the blood samples from the mammal showing specific antibody response to certain allergen by natural exposure to the allergen, (b) screening and selecting a mammal plasma having higher titers of allergen-specific immunoglobulin in the plasma with at least 2 fold higher serum dilution titer compared to normal mammals of same species, (c) purifying allergen-specific immunoglobulin from the mammal plasma containing high titers of allergen-specific immunoglobulin selected by the above step (b), (d) mixing the allergen-specific immunoglobulin purified in the above step (c) with histamine.

The present invention also provides a method of manufacturing a pharmaceutical composition for preventing or treating allergic diseases, which comprises the steps of (a) immunizing a mammal with an allergen, (b) measuring titers of allergen-specific immunoglobulin that can react with a certain allergen in the blood samples from the mammal, (c) screening and selecting a mammal blood having higher titers of allergen-specific immunoglobulin in the blood with at least 2 fold higher serum dilution titer compared to normal mammals of same species, (d) obtaining allergen-specific hyperimmune globulin by purifying immunoglobulin fraction containing allergen-specific immunoglobulin from the mammal blood having high titers of allergen-specific immunoglobulin selected by the above step (c), (e) mixing the allergen-specific hyperimmune globulin purified in the above step (d) with histamine.

The term “composition” as used hereinbefore or hereinafter in the specification of present invention is regarded as including any product formed by combination of specific ingredients directly or indirectly as well as a product containing the specific ingredients.

Each of ingredients used in the present composition can be present separately or complexly in the composition of the present invention, in the injectable formulation in which the present composition is dissolved, or in living bodies. For instance, histamine and antigen-specific antibody can form a complex in which they are bonded covalently or non-covalently.

The present composition includes a composition in which one of active ingredients is pharmaceutically or physiologically acceptable salt, a composition in which all of active ingredients are pharmaceutically or physiologically acceptable salts, a composition in which one of active ingredients is pharmaceutically or physiologically acceptable salt and the other ingredient(s) is free base form, or a composition in which the complex of one or more ingredients is pharmaceutically or physiologically acceptable salt.

The salts of the active ingredients or the complex of one or more ingredients in the present composition are meant to comprise all forms of pharmaceutically or physiologically acceptable salts. The pharmaceutically or physiologically acceptable salts of the active ingredients or the complex of one or more ingredients in the present composition includes water-soluble, oil-soluble or insoluble salt forms. For example, they include the conventional non-toxic salts or the quaternary ammonium salts which are formed, e.g., from organic or inorganic acids or bases.

An active ingredient, “histamine” in the composition of the present invention is a compound of formula C₅H₉N₃, which is broadly present within living bodies. It is formed from decarboxylation of histidine in protein by putrefactive bacterium or enteric bacterium. It is regarded that histamine is present in tissues as inactive form which is bonded with tissue protein, but when allergic reaction or anaphylaxis is developed, the inactive histamine become to be active form by certain action, thereby the activated histamine acts to organs or tissues. Histamine used in the present composition can be chemically prepared by well known methods in the art, or can be a selling good obtained from the art.

In an embodiment of the present invention, the term “allergen-specific antibody” indicates an antibody capable of specifically reacting with allergen that is defined as an antigen inducing an allergic reaction. In an embodiment of the present invention, the “allergen-specific antibody” can be an allergen-specific immunoglobulin.

In an embodiment of the present invention, the term “allergen-specific antibody” includes total antibody contained in the plasma obtained from mammal hyperimmunized with certain allergen. For example, “allergen-specific antibody” can indicate “allergen-specific hyperimmune globulin.”

“Allergen-specific hyperimmune globulin” indicates total immunoglobulin purified from blood of mammal immunized with certain allergen and thereby having high titers of allergen-specific antibody. For example, allergen-specific hyperimmune globulin indicates total immunoglobulin purified from bloods of mammals having higher titers of allergen-specific antibody in the blood at least 2 fold higher serum dilution titer compared to normal mammals of same species by immunizing certain allergen or by spontaneously. “Allergen-specific hyperimmune globulin” includes “allergen-specific antibody” or “allergen-specific immunoglobulin.” Therefore, “allergen-specific antibody” or “allergen-specific immunoglobulin” can be purified from “allergen-specific hyperimmune globulin” and can be used for the manufacture of pharmaceutical composition of the present invention.

Also, in the present invention, the above allergen-specific antibody can be immunoglobulin G (IgG) purified by physical method using the ability of allergen-specific antibody that can specifically bind to allergen.

The “allergen” in the present invention indicates all antigens which can induce allergic reaction or hypersensitivity reaction. Allergen that is defined as antigen inducing allergic reaction usually has a form of protein having antibody binding site (epitope).

In the present invention, allergen includes external allergen and internal allergen (autoallergen).

External allergen can be common materials such as mite, pollen, animal dander, fungus, food, synthetic fiber, accessory, drug, cosmetics, etc. Most of common materials contacted by eating, touching, breathing can be an allergen. External allergens can be broadly classified to inhalant allergen, food allergen, drug allergen, or contact allergen. Inhalant allergen means materials which were inhaled into living body during respiration. It can include pollen, house dust mite, animal (including dog, cat, ect.) dander, fungus, adhesive, paints, etc. Food allergen means materials which can induce hypersensitivity reaction or allergy reaction among eatable materials. It can include egg, milk, milk products, meat, bean, buckwheat, shrimp, crab, peach, processed food, etc. Drug allergen means materials which can induce hypersensitivity reaction or allergic reaction by getting into living body by injection or as an oral medication. It can include antibiotics, analgesics, hormone drugs, etc. Contact allergen means materials which can induce hypersensitivity reaction or allergy reaction by contacting to skin. It can include cosmetics, dye, clothing, detergent, rubber, metal, chemical materials, etc.

Internal allergen (autoallergen or autoantigen) includes nuclear antigen protein, double stranded DNA, phospholipid, beta-2 glycoprotein I, Fc fragment of human IgG antibody, or type II collagen previously shown to be target autoantigens of systemic lupus erythematosus and rheumatoid arthritis, but is not restricted to the above allergens.

Preferably, the allergens used in the present invention to obtain “allergen-specific antibody” can be, but are not limited to, pollen, house dust mite, animal dander, fungus or their mixtures which can frequently induce allergic reaction in many people. Preferably, patients with allergic diseases can be divided into several groups according to the kind of allergen (sensitized) showing allergic reaction in each patient, and then the allergen used in the present composition can be consisted of single allergen component or mixture of several allergen components suitable for the treatment of the allergic diseases in such patient groups. The causative allergen inducing allergic reaction to animal or human can be confirmed by serum allergen-specific IgE antibody test or skin test administrating allergen into skin according to known method to observe skin flare, wheal or edema (Board of Directors. Allergen skin testing. J Allergy Clin Immunol 92:653-7, 1993; Bierman C W, et al. (eds.) Allergy, asthma, and immunology from infancy to adulthood. p 144-156, Saunders, Philadelphia, 1996).

In a case that “allergen-specific antibody” of the present invention is “allergen-specific immunoglobulin,” “immunoglobulin” indicates a glycoprotein having important role in immunity and acting as antibody that can be restricted by the common characteristics in physical and structural properties, and common characteristics in the amino acid sequences. Basic structure of immunoglobulins consists of a pair of L chain (light chain) having molecular weight of about 23,000 and a pair of H chain (heavy chain) having molecular weight of about 50,000 to 70,000, wherein the L chain and H-chain are linked to each other by S-S bond. Immunoglobulin is classified as IgG, IgA, IgM, IgD, IgE according to the kinds of H chain, i.e., γ, α, μ, δ, ε. The immunoglobulin used in the present composition can be IgG, IgA, IgM, IgD, IgE or their mixture thereof, their fragments having biologically equal activity or the mixture of the fragments.

Also, the allergen-specific antibody used in the composition of present invention can be manufactured following example methods. Total immunoglobulin can be purified from plasma of mammals having significantly higher titers of allergen-specific antibody compared to normal mammals of same species by immunizing certain allergen or spontaneously due to natural exposure to the allergen. For purification of total immunoglobulin from the above mammal plasma, ethanol precipitation, ion-exchange affinity chromatography, or affinity chromatography using Protein A or Protein G columns can be used as currently commonly practiced in the associated fields. And the resulting purified total immunoglobulin of mammals having significantly high titers of allergen-specific antibody is hyperimmune globulin to specific allergen and this can be used as allergen-specific immunoglobulin of the present invention. And the allergen-specific antibody can be purified from the plasma of mammals having high titers of allergen-specific antibody by various methods like affinity chromatography using column containing agarose beads coupled with certain allergen as currently commonly practiced in the associated fields. And the allergen-specific antibody purified by the above methods can be used for the manufacture of the pharmaceutical composition of the present invention. The allergen-specific antibody also can be manufactured by genetic engineering techniques using cDNA library containing information on the antibody proteins obtained from peripheral blood mononuclear cells of mammals having high titers of allergen-specific antibody in their blood and by obtaining genetic information on the allergen-specific antibody from the cDNA library as widely known in the associated fields. The recombinant mammal immunoglobulin proteins prepared by the above genetic engineering technique can be further developed to humanized recombinant immunoglobulin changing the amino acid sequences of mammal immunoglobulin to amino acid sequences of human immunoglobulin (Vaughan T J, et al. Human antibody design. Nature Biotech 1998;16:535-539). The above allergen-specific antibody also can be fragments of immunoglobulin retaining biologically equal activity of allergen-binding portion like a F(ab)′2 or Fab fragments (Vaughan T J, et al. Human antibodies design. Nature Biotech 1998;16:535-539).

The antigen-specific antibody used in the composition of the present invention can be obtained from other species of animal different from the mammal to administrate the composition containing antigen-specific antibody. It is well known that immunoglobulin from other mammals can exert same pharmacological effects if it is administered to human because high degree of homology exists between the immunoglobulins from different species of mammals. Accordingly, the effects of preventing and treating allergic diseases by administrating the composition of the present invention can be operating in a same way even if the species of mammal who receive the composition of the present invention is different from the mammal produced the allergen-specific antibody.

The composition of the present invention can be manufactured by the following methods including the steps of (a) immunizing a mammal with allergen, (b) measuring titers of allergen-specific immunoglobulin in the blood samples from the mammal, (c) selecting mammal plasma having higher titers of allergen-specific antibody in the plasma with at least 2 fold higher serum dilution titer compared to normal mammals of same species, (d) purifying allergen-specific immunoglobulin from the mammal plasma containing high titers of allergen-specific immunoglobulin selected by the above step (c), (e) mixing the allergen-specific immunoglobulin purified in the above step (d) with histamine.

The composition of the present invention can be manufactured by the following methods including the steps of (a) measuring titers of allergen-specific immunoglobulin that can react with a certain allergen in the blood samples from the mammal showing specific antibody response to certain allergen by natural exposure to the allergen, (b) screening and selecting a mammal plasma having higher titers of allergen-specific immunoglobulin in the plasma with at least 2 fold higher serum dilution titer compared to normal mammals of same species, (c) purifying allergen-specific immunoglobulin from the mammal plasma materials containing high titers of allergen-specific immunoglobulin selected by the above step (b), (d) mixing the allergen-specific immunoglobulin purified in the above step (c) with histamine.

The composition of the present invention can be manufactured by the following methods including the steps of (a) immunizing a mammal with allergen, (b) measuring titers of allergen-specific immunoglobulin that can react with a certain allergen in the blood samples from the mammal, (c) screening and selecting a mammal blood having higher titers of allergen-specific immunoglobulin in the plasma with at least 2 fold higher serum dilution titer compared to normal mammals of same species, (d) obtaining allergen-specific hyperimmune globulin by purifying immunoglobulin fraction containing allergen-specific immunoglobulin from the mammal blood having high titers of allergen-specific antibody selected by the above step (c), (e) mixing the allergen-specific hyperimmune globulin obtained in the above step (d) with histamine.

To prepare the pharmaceutical compositions of this invention, the active ingredients can be combined in intimate admixture with a pharmaceutically acceptable carrier, which carrier may take a wide variety of forms depending on the form of preparation desired for administration. These pharmaceutical compositions are desirably in unitary dosage form, and can take dilutable form to control dosage depending on doctors' judgment.

The composition of the present invention is to use for subcutaneous injection. In embodiments of the present invention, however, the composition can be administered intravenously, intraarterially, intramuscularly, intraperitoneally, intrasternally, percutaneously, intranasally, rectally, orally, intraocularly, intradermally, locally, or by inhalation, according to ordinary methods.

Buffer for injection and other additive components to prepare the present composition to an injection formulation are well-known in the art. The injection formulation of the present composition can comprise additive components such as solubilizers, pH adjusting agents, suspending agents, etc., besides the buffer for injection. As the buffer for injection, physiological saline, etc. can be used.

The administration of composition of the present invention to mammal with allergic reaction to certain allergen shows an anti-allergic effect suppressing the allergen-specific IgE antibody response in the mammal as described in the examples of the present invention. The composition of the present invention not only suppresses the allergen-specific IgE antibody response but also additionally suppresses the allergen-specific IgG antibody response. Accordingly, the composition of the present invention can effectively prevent or treat allergic diseases that are known to be developed by IgE-mediated mechanism or IgG-mediated mechanism against certain allergens.

As described in the above, the composition of the present invention not only suppresses the allergen-specific IgE antibody response but also suppresses the allergen-specific IgG antibody response if administered to the patients with allergic diseases. The composition of the present invention can effectively prevent or treat the autoallergic diseases (or autoimmune diseases) if the “autoallergen (or autoantigen)-specific antibody” is included in the composition of the present invention as an “allergen-specific antibody” because the autoallergic diseases (or also called as autoimmune diseases) are known to be developed by IgG autoantibody-mediated mechanism.

Accordingly, the composition of the present invention can be used for treating allergic diseases. The examples of the above treatable allergic diseases due to external environmental allergens can include, but are not limited to, atopic dermatitis, allergic rhinitis, allergic conjunctivitis, urticaria or bronchial asthma. The examples of the above treatable allergic diseases that are developed by allergic reaction to internally existing self-antigens (also called as autoallergic reaction or autoimmune reaction) can include, but are not limited to, rheumatoid arthritis, systemic lupus erythematosus, and pemphigus.

Also, the present invention provides kits for preventing or treating allergic diseases comprising containers which contain active ingredient(s) of the pharmaceutical composition of the present invention separately or together. In an embodiment of the present invention, this invention provides a kit for preventing or treating allergic diseases comprising: a first container containing histamine; a second container containing allergen-specific antibody. Otherwise, the present invention provides a kit for prevention or treatment of allergic diseases comprising: first container histamine and allergen-specific antibody; and second container containing buffer for injection.

The present invention provides a use of the pharmaceutical composition comprising histamine and allergen-specific antibody as active ingredients for the manufacture of medicament for preventing or treating allergic diseases. The pharmaceutical composition of the present invention comprising histamine and allergen-specific antibody as active ingredients can be used for the manufacture of medicament for preventing or treating allergic diseases.

Also, the present invention provides a method for preventing or treating allergic diseases which comprises administrating a pharmaceutical composition comprising a therapeutically effective amount of histamine and allergen-specific antibody to a mammal.

The term “mammal” as used hereinbefore or hereinafter means mammal as a subject for treatment, observation or examination, preferably, human.

The term “therapeutically effective amount” used hereinbefore or hereinafter means that amount of active ingredient or pharmaceutical composition that elicits the biological or medicinal response in a tissue, system, animal or human by a researcher, veterinarian, medical doctor or other clinician, which can induce alleviation of the symptoms of the disease or disorder being treated.

The method for preventing or treating allergic diseases in the present invention can be performed by using the above pharmaceutical composition.

The dosage of the pharmaceutical composition in the present invention can be decided by considering the dosage of histamine and immunoglobulin used in the treatment using histamine-immunoglobulin complex. Though the dosage of general pharmaceutical composition can be decided depending on severity of the clinical symptoms, age, weight of the patient, etc., the dosage of the present composition should be decided by considering patient's sensitivity for the allergen causing the allergic diseases and/or patient's sensitivity for histamine or immunoglobulin, as well as the above condition.

When the pharmaceutical composition of the present invention comprising histamine and antigen-specific antibody is administrated at once, a dosage of histamine can be 0.05 to 2.5 μg, preferably 0.1 to 1.0 μg, and more preferably 0.15 to 0.45 μg. Also, when the pharmaceutical composition is administrated at once, a dosage of immunoglobulin can be 0.01 to 50 mg, and preferably 12 to 36 mg. Preferably, histamine and allergen-specific antibody can be combined and then dissolved in buffer for injection of 0.5 to 2 ml at once.

Preferably, histamine, allergen-specific antibody, and other additive components are provided in the forms of lyophilized powder sealed separately, and then dissolved to use before administration according to the doctor's decision for the dosage depending on condition of the patient.

The dosage of the above active ingredients at a time of treating allergic diseases is not fixed, and can be increased gradually considering the sensitivity of the patients to the first administration dose. Also, a dosage of the pharmaceutical composition of the present invention can be controlled by the doctor's decision with wide experience considering the patient's condition according to the administration of the composition of the present invention.

It is evident for a skilled artisan that the therapeutically effective amount of the active ingredients and the pharmaceutical composition containing the active ingredients of the present invention and the number of their administration will be varied depending on desirable effect. Therefore, the most suitable dosage to be administrated can be decided easily, and it can be varied depending on certain active ingredient to be used, mode of administration, effect of formulation and development of the diseases condition. Also, it will be needed to control the dosage of administration to adjust treatment level appropriately depending on patients' individual factors including age, body weight, diet, and timing of administration, etc.

The advantages and features of the present invention and the method of revealing them will be explicit from the following examples described in detail. However, it is to be distinctly understood that the present invention is not limited thereto but may be otherwise variously embodied and practiced. It is obvious that the following examples are to complete the disclosure of the invention and to indicate the scope of the present invention to a skilled artisan completely, and the present invention will be defined only by the scope of the claims.

### BRIEF DESCRIPTION OF THE DRAWING

FIG. 1 shows the result of measuring titers of specific IgG antibody to ovualbumin (OVA) in the serum samples of 3 rabbits immunized with OVA (OVA-1, OVA-2, OVA-3) and 2 normal rabbits (normal control 1, normal control 2) by enzyme-linked immunosorbent assay (ELISA).

FIG. 2 shows the result of measuring titers of specific IgG antibody to ovualbumin (OVA) by enzyme-linked immunosorbent assay (ELISA) in the immunoglobulin samples purified from one rabbit (OVA) immunized with OVA and one normal rabbit (normal control) using Protein A column.

FIG. 3 shows the result of measuring titers of specific IgG antibody to ovualbumin (OVA) in the commercially available human immunoglobulin formulations including immunoglobulin for intramuscular injection (NL/Ig) and histamine-immunoglobulin complex (His/Ig) and in the immunoglobulin purified from blood of a patient suffering from allergic disease (Patient 1) by enzyme-linked immunosorbent assay (ELISA).

FIG. 4 shows the result of measuring titers of specific IgG antibody to house dust mite in the commercially available human immunoglobulin formulations including immunoglobulin for intramuscular injection (NL/Ig) and histamine-immunoglobulin complex (His/Ig) and in the immunoglobulin purified from blood of a patient suffering from allergic disease who had been treated by allergen-immunotherapy with house dust mite allergen due to allergic asthma and allergic rhinitis (Patient 1) by enzyme-linked immunosorbent assay (ELISA).

### MODE FOR THE INVENTION

Examples of Formulation

Example of Formulation 1:

Allergen-specific antibody 12 mg

Histamine dichloride 0.15 μg

Sodium chloride 4 mg

Amino acetic acid 45 mg

D-manitol 4 mg

Sodium hydroxide—an appropriate amount

Solution for injection 0.8-2 ml (supplied as another vial from the above ingredients)

Example of Formulation 2:

Allergen-specific hyperimmune globulin 12 mg

Histamine dichloride 0.15 μg

Sodium chloride 4 mg

Amino acetic acid 45 mg

D-manitol 4 mg

Sodium hydroxide—an appropriate amount

Solution for injection 0.8-2 ml (supplied as another vial from the above ingredients)

### EXAMPLE I

Suppression of Allergen-Specific IgE Antibody-Mediated Allergic Reaction and Allergen-Specific IgG Antibody-Mediated Immunologic Reaction by an Administration of a Composition Comprising Histamine and Allergen-Specific Antibody.

I-1. Manufacture of Allergen-Specific Antibody.

I-1-1) Immunization with Ovualbumin (OVA)

To produce allergen-specific immunoglobulin that will be used for the manufacture of a histamine/allergen-specific antibody complex, 3 rabbits were immunized by subcutaneous injection of 100 μg of OVA mixed with complete Freund's adjuvant at first day and subcutaneous injections of 100 μg of OVA mixed with incomplete Freund's adjuvant at 3 week and at 6 week. Female rabbits with New Zealand White species, with 3 months-old age, and body weight ranging from 2.2-2.5 kg were used in the above experiment.

I-1-2) Measuring Titer of OVA-Specific Antibody and Selecting Blood Samples with High Titer of OVA-Specific Antibody.

Blood was sampled from the above OVA-immunized rabbits at 9 weeks after the first OVA-immunization (3 week after the last subcutaneous injection of OVA) and serum samples were obtained from the blood samples. And rabbit serum samples having high titers of OVA-specific antibody were screened by enzyme-linked immunosorbent assay (ELISA). The measurement of OVA-specific IgG antibody in rabbit serum samples was performed as the same method of measuring OVA-specific IgG antibody in mouse serum samples described in example I-2 of the present invention except for using alkaline phosphatase conjugated anti-rabbit IgG antibody as a secondary conjugate. Because of the expectation on a high variability in titers of OVA-specific immunoglobulin among the individual rabbits, the serum samples were serially diluted starting from 1:10,000 dilutions with 10-fold dilutions between samples to measure the titers of OVA-specific antibody.

The positive detection of OVA-specific IgG antibody was defined as when the absorbance value of tested rabbit serum sample was greater than mean +3 standard deviation of absorbance values of normal rabbit serum samples (not immunized with OVA) and when the absorbance value of tested rabbit serum sample was greater than 2 fold of mean of absorbance values produced by dilution buffer (phosphate buffered saline containing 3% bovine serum albumin) (the absorbance values of dilution buffer were less than 0.01 in the examples of the present invention). Accordingly, the titer of OVA-specific IgG antibody was expressed as a highest serum dilution or concentration of purified immunoglobulin showing positive detection of OVA-specific IgG antibody.

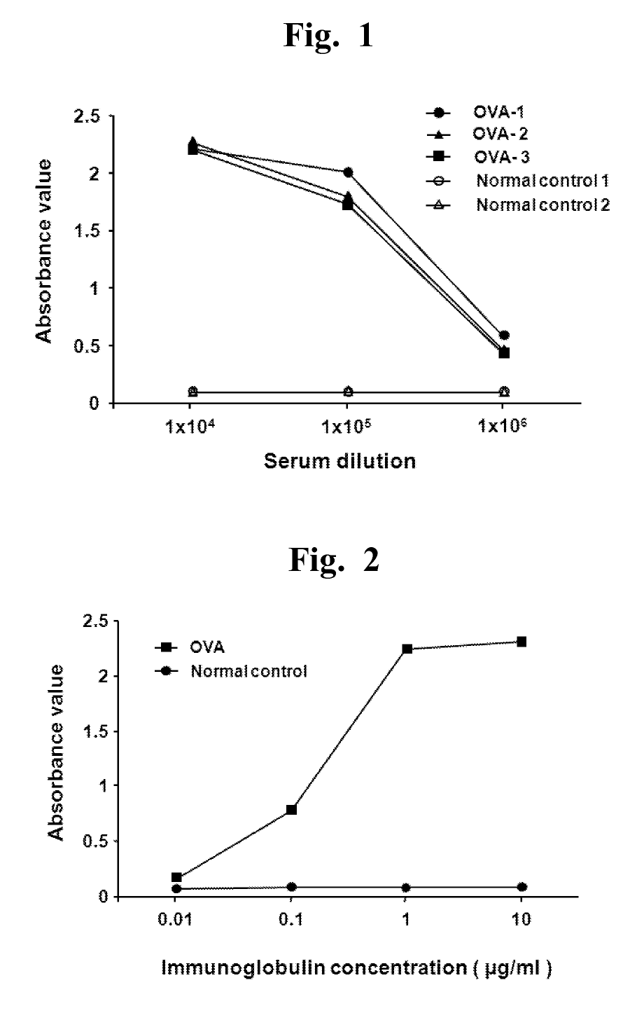

The serum samples from 3 rabbits immunized with OVA had high titers of OVA-specific IgG antibody (at least 1:1,000,000 serum dilutions) showing significantly higher absorbance values compared to the absorbance values of the serum samples from normal rabbits (negative controls) or absorbance values of dilution buffer (phosphate buffered saline containing 3% bovine serum albumin) (the absorbance values of dilution buffer were less than 0.01 in the examples of the present invention) (FIG. 1). The OVA-specific IgG antibody was not detected in the serum samples from 2 normal rabbits without a prior OVA immunization even at the 1:10,000 serum dilutions (FIG. 1). The OVA-specific IgG antibody was positively detected even in the 0.1 μg/ml concentration of immunoglobulin purified from a serum sample of OVA-immunized rabbit using a Protein A column as described in the example I-1-3 of the present invention (FIG. 2). However, the OVA-specific IgG antibody was not detected even in the 10 μg/ml concentration of immunoglobulin purified from serum sample of a normal rabbit (negative control) by the same method showing absorbance values that were not significantly different from the mean absorbance value of dilution buffers (the absorbance values of dilution buffer were less than 0.01) (FIG. 2).

Accordingly, the above results show that the mammal serum samples having high titers of allergen-specific immunoglobulin with at least 2 fold higher serum dilution titer by hyper-immunization with allergen (ex, OVA) compared to the serum samples from normal mammals of same species without prior allergen immunization can be effectively screened and selected by the simple assays measuring titers of allergen-specific antibody (ELISA, etc) (FIG. 1). And the allergen-specific hyperimmune globulin (that can be defined as immunoglobulin purified from a mammal serum sample having high titer of allergen-specific antibody by immunization with allergen such as OVA) still shows high titer of allergen-specific immunoglobulin with at least 2 fold higher titer compared to the immunoglobulin purified from a normal mammal serum without prior allergen immunization (FIG. 2).

Accordingly, the serum samples of rabbits having high titer of OVA-specific antibody in the blood were screened by the above assays measuring OVA-specific antibody and were used for the manufacture of a composition comprising histamine and allergen-specific hyperimmune globulin in the below example I-1-3. The above example demonstrates that allergen-specific antibody or allergen-specific hyperimmune globulin contained in the pharmaceutical composition of the present invention can be clearly differentiated from the histamine-immunoglobulin complex containing immunoglobulin purified from normal mammal without prior allergen immunization by simple assays measuring titers of allergen-specific antibody.

I-1-3) Purification of Allergen-Specific Hyperimmune Globulin and Manufacture of a Histamine/Allergen-Specific Hyperimmune Globulin Complex

IgG was purified using affinity chromatography with protein A column from the rabbit serum sample demonstrated to have high titer of OVA-specific antibody by the above ELISA assay and the purified IgG was used as a OVA-specific hyperimmune globulin and allergen-specific antibody of the present invention. IgG was purified from serum samples of two rabbits without prior OVA immunization and these IgG were used as negative control normal rabbit immunoglobulin. The above purified rabbit IgG samples were concentrated to an appropriate concentration and changed the buffer with normal saline using centrifugal filtration device (Centricon™; Milipore, U.S.A.). The titers of OVA-specific IgG antibody in the purified IgG were reconfirmed by ELISA after measuring the immunoglobulin concentration in the sample of purified IgG (FIG. 2). A histamine/OVA-specific hyperimmune globulin complex was manufactured by mixing 12 mg of the purified rabbit OVA-specific hyperimmune globulin dissolved in normal saline and 0.15 μg of histamine dichloride and incubating for 2 hours at room temperature. As a negative control, histamine/normal immunoglobulin complex was manufactured by mixing 12 mg of the purified normal rabbit immunoglobulin and 0.15 μg of histamine dichloride and incubating for 2 hours at room temperature.

I-2. Development of Ovualbumin (OVA) Allergy Mouse Model

Allergic diseases are known to be developed as results of release of various chemical mediators including histamine and TNF-alpha from cells bearing high affinity IgE receptor (basophil or mast cell) induced by binding of allergen with allergen-specific IgE antibody existing on the surface of the above cells regardless the kinds of allergens (Platts-Mills T A. Am J Respir Crit Care Med 2001;164:S1-5).

Accordingly, ovualbumin (OVA) allergy mouse model was used for the demonstration of anti-allergic effects of histamine/allergen-specific immunoglobulin complex of the present invention because the OVA allergy mouse model has been most commonly used for the demonstration of anti-allergic effects in the developments of new anti-allergic drugs in the related field of art (Ayoub M, et al. Int Immunopharmacol 2003;3:523-53; Horner A A, et al. J Allergy Clin Immunol 2002;110:413-20; Allergen compostion. Korean Patent No. 10-0549557). OVA-allergy animal model was used in the examples of the present invention to demonstrate the pharmaceutical efficacy because it is known that OVA allergy animal model is very easy, inexpensive, highly reproducible, and no significant difference compared to the allergy animal model using other allergens as widely known to the skill artisan in the current industrial and academic fields.

Six week-old female BALB/c mouse was used for the development of OVA allergy mouse model. Each mice were received mixtures of 20 μg of OVA (Sigma Chemical Co., St. Louis, Mo.) and adjuvant containing 2 mg of aluminum hydroxide and 2 mg of magnesium hydroxide (Alumn™, Pierce, Rockford, Ill.) by intraperitoneal injections at day 0 and day 14. A degree of allergic immune response in the mouse was evaluated by measuring OVA-specific IgE and IgG antibodies in the sera sampled from the tail of mouse at day 14, day 21, or day 28.

Specific IgE antibody to OVA were measured with enzyme-linked immunosorbent assay (ELISA) by the previously described method with modification (Saloga J, et al. J Clin Invest 1993;91:133-40). Ninety six-well microtiter plates were coated with 5 μg of OVA per well and blocked the nonspecific bindings by incubating with phosphate buffered saline (PBS) containing 0.1% TWEEN-20™ and 3% bovine serum albumin for 1 hour. The mouse serum samples were diluted with the above blocking buffer at 1:20 (v/v) dilution and applied to the above wells and incubated for 3 hours. Then, the above wells were incubated with biotin-labeled anti-mouse IgE antibody (rat monoclonal antibody) for 1 hour and then incubated with alkaline phosphatase conjugated streptavidin for 30 minutes. Then, a degree of antigen-antibody reaction was measured by color development with p-nirophenyl phosphate. The above ELISA plate was washed 5 times with PBS containing 0.1% TWEEN-20™ (PBST) between each reaction steps. Normal mouse serum samples without prior OVA immunization were used as negative controls. The results were expressed as absorbance values measured at 405 nm. Mean absorbance values from duplicated measurements of each individual samples were used as results of experiments.

I-3. Suppression of OVA-Specific IgE and OVA-Specific IgG Antibody Response by an Administration of the Histamine/OVA-Specific Hyperimmune Globulin Complex

OVA allergy mouse model developed as the above example I-2 was treated with histamine/OVA-specific hyperimmune globulin manufactured by the above example I-1-3. OVA allergy mouse model was developed in total 24 mice by injection of OVA as the above example I-2 and these mice were divided into 3 groups for experiments. Mice of group 1 (a normal saline treated negative control group, n=8) received subcutaneous injections of 100 μl of normal saline at day 1 and day 15. Mice of group 2 (n=8) received subcutaneous injection of a histamine/OVA-specific hyperimmune globulin complex (His/OHIgC) 2 mg (as an immunoglobulin content) manufactured by the above example I-1-3 at day 1 and day 15. Mice of group 3 (n=8) received subcutaneous injection of a histamine/normal rabbit immunoglobulin complex 2 mg (as an immunoglobulin content) manufactured by the above example I-1-3 at day 1 and day 15.

OVA-specific IgE and IgG antibodies were measured in the sera sampled from the tail of the above mice at day 21 of the experiment. The analyzed results were shown in the below Table 1 (OVA-specific IgE) and table 2 (OVA-specific IgG).

TABLE 1
Suppression of OVA-specific IgE antibody response by a
histamine/OVA-specific hyperimmune globulin complex.

| | | Levels of OVA-specific | *p-value | *p-value |
|---|---|---|---|---|
| | | IgE antibody | when | when |
| | Numbers | (mean ± S.D. of | compared | compared |
| Experiment groups | of mice | absorbance values) | with group 1 | with group 2 |
| Group 1- | 8 | 0.495 ± 0.189 | | 0.003 |
| Normal saline treated | | | | |
| group | | | | |
| Group 2- | 8 | 0.197 ± 0.024 | 0.003 | |
| His/OHIgC treated | | | | |
| group | | | | |
| Group 3- | 8 | 0.907 ± 0.310 | 0.006 | <0.001 |
| His/NRIgGC treated | | | | |
| group | | | | |

S.D. = standard deviation;
*p-value was calculated by comparing mean values between two groups by Student's t-test (independent sample test) and p value < 0.05 was considered as statistically significantly difference.
His = histamine dichloride,
His/OHIgC = histamine and ovualbumin-specific hyperimmune globulin complex,
His/NRIgGC = histamine and normal rabbit IgG complex.

There were statistically significant differences in the mean values of OVA-specific IgE levels among the above 3 groups (Table 1, one-way Anova test, p<0.001). Levels of OVA-specific IgE antibody were significantly decreased in the group 2 mice treated by a histamine/OVA-specific hyperimmune globulin complex compared to the group 1 mice treated by normal saline or groups 3 mice treated by a histamine/normal rabbit immunoglobulin complex (Table 1, Student's t-test, p<0.05). This result confirms that histamine/OVA-specific hyperimmune globulin complex of the present invention can statistically significantly suppress the specific IgE antibody response to allergen (Table 1). Interestingly, levels of OVA-specific IgE were significantly increased in the group 3 mice treated by a histamine/normal rabbit immunoglobulin complex compared to group 1 mice treated by normal saline (Table 1, p<0.01).

There were statistically significant differences in the mean values of OVA-specific IgG levels among the above 3 groups as shown the below Table 2 (one-way Anova test, p<0.001).

TABLE 2
Suppression of OVA-specific IgG antibody response by a
histamine/OVA-specific hyperimmune globulin complex.

| | | Levels of OVA-specific | *p-value | *p-value |
|---|---|---|---|---|
| | | IgG antibody | when | when |
| | Numbers | (mean ± S.D. of | compared | compared |
| Experiment groups | of mice | absorbance values) | with group 1 | with group 2 |
| Group 1- | 8 | 0.666 ± 0.244 | | <0.001 |
| Normal saline treated | | | | |
| group | | | | |
| Group 2- | 8 | 0.180 ± 0.030 | <0.001 | |
| His/OHIgC treated | | | | |
| group | | | | |
| Group 3- | 8 | 0.564 ± 0.326 | 0.489 | 0.013 |
| His/NRIgGC treated | | | | |
| group | | | | |

S.D. = standard deviation;
*p-value was calculated by comparing mean values between two groups by Student's t-test (independent sample test) and p value < 0.05 was considered as statistically significantly difference.
His = histamine dichloride,
His/OHIgC = histamine and ovualbumin-specific hyperimmune globulin complex,
His/NRIgGC = histamine and normal rabbit IgG complex.

Levels of OVA-specific IgG antibody were significantly decreased in the group 2 mice treated by histamine/OVA-specific hyperimmune globulin complex compared to the group 1 mice treated by normal saline or groups 3 mice treated by histamine/normal rabbit immunoglobulin complex (Table 2, Student's t-test, p<0.05). This result confirms that histamine/OVA-specific hyperimmune globulin complex of the present invention can statistically significantly suppress the antigen-specific IgG antibody response to OVA (Table 2).

A critical central role of allergen-specific IgE antibody response in the development of allergic diseases has been demonstrated with the significant improvements of clinical symptoms with suppression of specific IgE antibody-mediated allergic immune response to causative allergens by the below 3 methods. 1) avoidance of enviromenal allergen, 2) allergen-specific immunotherapy, and 3) anti-IgE antibody therapy (Platts-Mills T A. Am J Respir Crit Care Med 2001;164:S1-5). Furthermore, allergic response to specific allergen induced in a mammal and physiological changes resulting from allergic response to specific allergen in the mammal can be reproduced in the other mammal by passive transfer of allergen-specific IgE antibody to the other mammal (Oshiba A, et al. J Clin Invest 1996;97:1398-408). And the above experiment also reported a possible partial contribution of allergen-specific IgG antibody in the development of allergic disease (Oshiba A, et al. J Clin Invest 1996;97:1398-408).

In the above example I-3, the present inventors demonstrated a marked anti-allergic efficacy of histamine/allergen-specific hyperimmune globulin complex by showing that histamine/allergen-specific hyperimmune globulin could suppress the allergen-specific IgE antibody response and allergen-specific IgG antibody response more significantly and more effectively than a currently used histamine/normal immunoglobulin complex for the first time. Until now, there has been no report on the association between antigen-reacting characteristics of immunoglobulin contained in the histamine/immunoglobulin complex and anti-allergic pharmaceutical action. And there has been no trial to improve an anti-allergic efficacy of histamine/immunoglobulin complex by utilizing the antigen-specificity of immunoglobulin contained in the pharmaceutical composition of histamine/immunoglobulin complex.

Accordingly, the example I-3 demonstrates that a pharmaceutical composition of the present invention comprising histamine and allergen-specific antibody as active ingredients designed by the present inventors shows very excellent anti-allergic effect that can be applied for treating allergic diseases including allergic asthma, allergic rhinitis, allergic conjunctivitis, and atopic dermatitis.

### EXAMPLE 2

Demonstrating the Efficacy of a Pharmaceutical Composition of the Present Invention Comprising Histamine and Allergen-Specific Antibody Complex by Proving Reduction of Anti-Allergic Effect of Currently Existing Formulation of Histamine-Immunoglobulin Complex After Removing Allergen-Specific Immunoglobulin from the Histamine-Immunoglobulin Complex

In this example, the anti-allergic efficacy of histamine-human immunoglobulin complex treatment after removing a complex of histamine-allergen-specific antibody from histamine-human immunoglobulin complex currently used for treating allergic diseases was compared with a histamine-human immunoglobulin complex treatment, human immunoglobulin treatment, and simultaneous treatment with histamine and human immunoglobulin in the OVA allergy mouse model and measuring the suppressing effects on the allergen-specific IgE antibody and IgG antibody mediated immune response.

II-1. Analysis of Materials for Experiment

A commercially available injection formulation of histamine-immunoglobulin complex (Histobulin™, Green cross Co., Korea) contained 12 mg of human immunoglobulin and 0.15 μg of histamine dichloride as described in the product information sheet provided by the manufacturer. The above formulation is provided as two vials comprising one vial containing the above active ingredients in a lyophilized form and another vial containing 2 ml of distilled water for injection and the manufacturer recommends to mix the contents of two vials using an injection syringe immediately before the each injections and dissolve the active ingredients well and inject 2 ml of this mixture subcutaneously. When the contents of IgG, IgM, IgA and albumin concentration in the above histamine-immunoglobulin complex injection solution were determined by nephelometry analyzer (COBAS INTEGRA, Roche Diagnostics GmbH, Germany), the 11.0 mg of human IgG and 0.24 mg of human IgA were contained in the above formulation, but IgM or albumin was not detected in the above formulation because their concentrations were below the lowest detection limits of the above analyzer (IgM<0.037 mg/ml, albumin<0.09mg/ml). And a commercially available injection solution of human immunoglobulin for intramuscular administration (Gamma globulin™; Green cross Co., Korea; contains 165 mg/ml of human gammaglobulin according to the product information provided by manufacturer; nephelometric anaysis of this product showed that the product contained IgG 150 mg/ml, IgA 0.14 mg/ml, IgM<0.04 mg/ml, and albumin 1.58 mg/ml) was used as a negative control material.

II-2. Measuring the Titers of Allergen-Specific Antibody Contained in an Injection Formulation of Histamine-Human Immunoglobulin Complex, a Formulation of Immunoglobulin for Intramuscular Injection, and the Immunoglobulin Purified from Human who had been Immunized with Allergen.

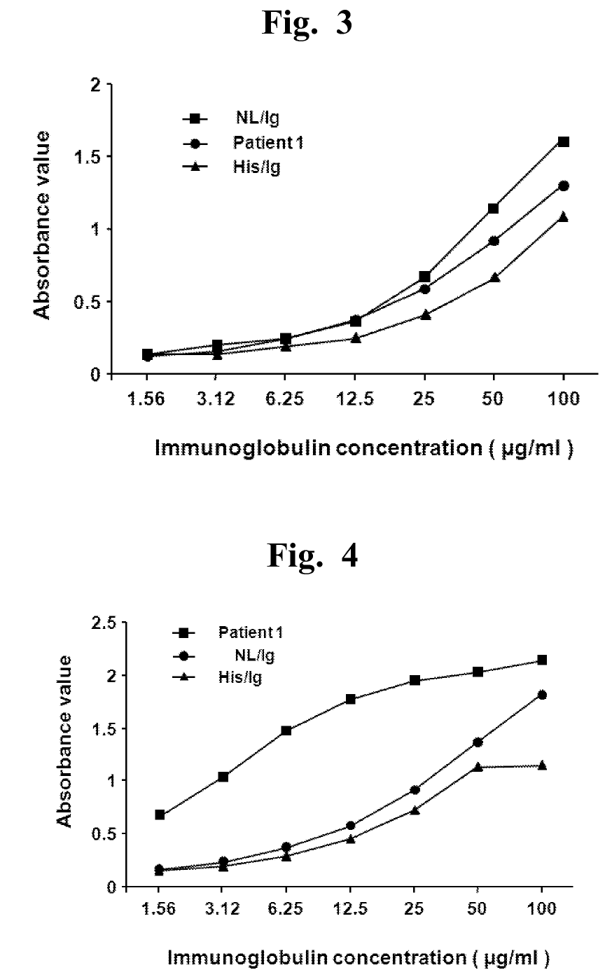

The present inventors could confirm the presences of significant amounts of allergen-specific IgG antibody in a commercially available injection formulation of histamine/human immunoglobulin complex and a formulation of immunoglobulin for intramuscular injection that were made from human plasma of multiple donors (FIG. 3, FIG. 4) as reported in the previous studies (Vance G H S, et al. Clin Exp Allergy 2004;34:1855-61; Stewart G A, et al. Clin Allergy 1988;18:235-43; Saint-Remy J M, et al. Allergy 1988;43:338-47) by measuring IgG antibody specific to OVA and house dust mite (Dermatophagoides farinae) allergens with ELISA method. The measurement of allergen-specific IgG antibody in the aboves used the same method of measuring OVA-specific IgG antibody in mouse serum samples described in example I-2 of the present invention except for using alkaline phosphatase conjugated anti-human IgG antibody as a secondary conjugate.

Interestingly, there were no significant differences of titers of IgG antibody to the above two kinds of allergens (OVA and house dust mite) between commercially available formulations of histamine-immunoglobulin complex and immunoglobulin for intramuscular injection when the titers of allergens-specific IgG antibody in the two formulations were compared after dilutions to the same concentrations of immunoglobulin (table 3, table 4). Accordingly, the above results of experiments in example II-2 demonstrates that the commercially available current formulation of histamine-immunoglobulin complex did not perform any special selection process of plasma materials concerning on the points of specific IgG antibody to common major allergens when compared with other immunoglobulin formulation for intramuscular injection (FIG. 3 and FIG. 4). Immunoglobulin was purified from blood sample of a patient with allergic rhinitis and house dust mite allergy who had been treated with allergen-specific immunotherapy receiving subcutaneous injections of house dust mite allergen for past 1 year (patient 1 in FIG. 3 and FIG. 4). The immunoglobulin from the above patient who received injections of house dust mite allergen showed high tiers of house dust mite-specific IgG antibody with at least 2 fold higher immunoglobulin concentration titers than the above two commercially available immunoglobulin formulations when the house dust mite-specific antibody was measured in two fold serial dilutions of immunoglobulin concentrations from 100 microgram/ml to 1.56 microgram/ml (FIG. 4). The specific IgG antibody to house dust mite allergen in the immunoglobulin from the above patient (patient 1 in FIG. 4) was positively detected even in the immunoglobulin concentration of 1.56 microgram/ml (FIG. 4). Accordingly, the above results of example II-2 confirm that allergen-specific hyperimmune globulin can be obtained from human subject if the human subject was immunized with certain allergen. And the above example demonstrates that allergen-specific antibody or allergen-specific hyperimmune globulin of the pharmaceutical composition of the present invention containing high titers of allergen-specific antibody obtained by immunizing mammal with certain allergen can be clearly distinguished from both immunoglobulin obtained from normal mammal or current formulation of histamine-immunoglobulin complex containing immunoglobulin with uncertain information on the antibody to specific allergen by simply measuring allergen-specific antibody with assay method as described in the above example. As shown in the above example, titers of allergen-specific antibody contained in the blood samples of normal blood donors were very low compared to the titer of allergen-specific antibody in the blood sample from a human subject hyperimmunized with allergen. And present inventors judge that the above factor about low titers of allergen-specific antibody in commercially available immunoglobulin formulation results in a current situation that relatively low proportion of patients with allergic diseases are experiencing clinical improvement by the treatment with currently used histamine-immunoglobulin complex and the therapeutic efficacy of currently used histamine-immunoglobulin complex in patients with allergic diseases are relatively minimal. Therefore, a pharmaceutical composition of the present invention comprising a complex of histamine and allergen-specific hyperimmune globulin that is prepared from the mammal immunized with allergen containing higher titers of allergen-specific antibody than normal mammals without prior allergen immunization will evidently show excellent efficacy for preventing and treating allergic disease as demonstrated in the below examples using animal model of allergy.

II-3. Removal of Allergen-Specific Antibody from Currently used Formulation of a Histamine-Human Immunoglobulin Complex

The present inventors hypothesized that low doses of allergen-specific antibody existing in the commercially available currently histamine-immunoglobulin complex have a critical role for the anti-allergic efficacy of the above formulation. The present inventors demonstrated the above hypothesis for the first time by the below experiments. The present inventors tried to develop a significantly advanced new formulation compared to current formulations on the basis of newly identified scientific principle that can overcome a disadvantage resulted from an uncertainty about the antigen-specificity of immunoglobulin in the currently available histamine-immunoglobulin complex.

To use as major materials in the experiments confirming the effects of OVA-specific antibody existing in the injection formulation of histamine-immunoglobulin complex, purified OVA (Sigma Chemical Co., St. Louis, Mo.) or human serum albumin (HSA; Green cross Co., Korea) were coupled to the cyanogens-bromide activated agarose beads (Sepharose 4B™, Sigma Chemical Co., St. Louis, Mo.) according to the recommendation of manufacturer of the agarose beads with ratio of 5 mg of OVA or HSA to 1 ml of activated agarose beads.

A 4 ml solution containing 10 mg/ml of histamine-human immunoglobulin complex was mixed with either 4 ml of OVA-coupled agarose beads or HSA-coupled agarose beads and the mixture was incubated for 16 hours at 4° C. and then the supernatants were separated. OVA-specific human IgG antibody was not detected in the above supernatant of histamine-immunoglobulin complex adsorbed by OVA-coupled agarose bead on ELISA anaysis. However, the levels of OVA-specific human IgG antibody in the above supernatant of histamine-immunoglobulin complex adsorbed by HSA-coupled agarose bead were not statistically significantly different compared to the levels of OVA-specific human IgG antibody in the original solution of histamine-human immunoglobulin complex on ELISA anaysis.

II-4. Suppression of Allergic Immune Response by a Histamine/Human Allergen-Specific Antibody Complex in an Animal Model of Allergy

To demonstrate a biological effect of histamine/allergen-specific antibody complex of present invention, OVA allergy was induced in 48 female BALB/c mice with the same methods described in the above example I-2. These 48 mice were divided into 6 groups (each groups consisted of 8 mice) and underwent below 6 different kinds of treatments along with OVA allergy induction.

Mice of group 1 (a normal saline treated negative control group) received subcutaneous injections of 100 μl of normal saline at day 1, 6, 10, 17, and day 24. Mice of group 2 (histamine/human immunoglobulin complex treated group) received subcutaneous injections of a histamine/human immunoglobulin complex 3 mg per mouse at day 1, 6, 10, 17, and day 24. Mice of group 3 (histamine/human immunoglobulin complex depleted of OVA-specific immunoglobulin treated group) received subcutaneous injections of 3 mg per mouse of a histamine/human immunoglobulin complex that depleted OVA-specific antibody by prior incubation with OVA-coupled agarose beads at day 1, 6, 10, 17, and day 24. Mice of group 4 (histamine/human immunoglobulin complex incubated with HSA-coupled agarose beads treated group) received subcutaneous injections of 3 mg per mouse of a histamine/human immunoglobulin complex supernatant incubated with HSA-coupled agarose beads at day 1, 6, 10, 17, and day 24. Mice of group 5 (human immunoglobulin for intramuscular injection treated group) received subcutaneous injections of 3 mg per mouse of a human immunoglobulin for intramuscular injection (example II-2) at day 1, 6, 10, 17, and day 24 as a negative control experiment of the above group 2 treatment. Mice of group 6 (histamine and human immunoglobulin for intramuscular injection treated group) received subcutaneous injections of 3 mg per mouse of a immediate mixture of histamine and human immunoglobulin for intramuscular injection with the same histamine/immunoglobulin ratio in a commercially available current histamine/human immunoglobulin complex formulation (example II-2) at day 1, 6, 10, 17, and day 24.

Levels of OVA-specific IgE antibody were measured in the sera sampled from the tail of the above mice at day 28 of the experiment. The analyzed results were shown in the below table 3.

TABLE 3
Suppression of allergic immune response by a histamine/human
OVA-specific antibody complex in OVA allergy mouse model

| | | Levels of | | |
|---|---|---|---|---|
| | | OVA-specific IgE | *p-value | *p-value |
| | | antibody | when | when |
| Experiment | Numbers of | (mean ± S.D. of | compared | compared |
| groups | mice | absorbance values) | with group 1 | with group 2 |
| Group 1- | 8 | 0.942 ± 0.236 | | 0.002 |
| Normal saline | | | | |
| treated group | | | | |
| Group 2- | 8 | 0.564 ± 0.138 | 0.002 | |
| His/hIg treated | | | | |
| group | | | | |
| Group 3- | 8 | 1.000 ± 0.352 | 0.702 | 0.010 |
| OVA-adsorbed | | | | |
| His/hIg treated | | | | |
| group | | | | |
| Group 4- | 8 | 0.451 ± 0.111 | <0.001 | 0.091 |
| HSA-adsorbed | | | | |
| His/hIg treated | | | | |
| group | | | | |
| Group 5- | 8 | 0.908 ± 0.319 | 0.812 | 0.020 |
| Human | | | | |
| immunoglobulin | | | | |
| treated group | | | | |
| Group 6- | 8 | 0.619 ± 0.117 | 0.006 | 0.411 |
| Human | | | | |
| immunoglobulin + | | | | |
| histamine treated | | | | |
| group | | | | |

S.D. = standard deviation;
*p-value was calculated by comparing mean values between two groups by Student's t-test (independent sample test) and p value < 0.05 was considered as statistically significantly difference.
His = histamine dichloride,
hIg = human immunoglobulin,
OVA = ovualbumin,
HSA = human serum albumin.

There were statistically significant differences in the mean values of OVA-specific IgE levels among the above 6 groups (Table 3, one-way Anova test, p<0.001). Levels of OVA-specific IgE antibody were significantly decreased in the group 2 mice treated by histamine/human immunoglobulin complex, in group 3 mice treated by histamine/human immunoglobulin complex adsorbed with HSA-coupled beads, and in group 6 mice treated by a immediate mixture of histamine and human immunoglobulin for intramuscular injection compared to the negative control group mice (group 1) treated by normal saline only (Table 3, p<0.05). The above results indicate that administration of histamine/human immunoglobulin complex can significantly suppress the allergen-specific IgE response (Table 3).

Human immunoglobulin used for the manufacture of commercially available histamine-immunoglobulin complex is purified from a mixture of plasma materials (pooled plasma) donated by multiple healthy volunteers through processing of well known art in the related field.

The presence of considerable amounts of specific antibody to OVA in the blood of normal subjects due to sensitization through food intakes has been reported by a previous study (Vance G H S, et al. Clin Allergy 2004;34:1855-61). Accordingly, the present inventors hypothesized that OVA-specific antibody contained in the histamine-human immunoglobulin complex suppress OVA-specific IgE antibody response in OVA allergy mouse model and logically demonstrated the following facts described in the below through the above experiments of example II-4.

Firstly, the presences of considerable amounts of specific IgG antibody to common food allergen like OVA or common inhalant allergen like house dust mite in the commercially available immunoglobulin formulations were confirmed in experimental results of the above example II-2 (FIG. 3, FIG. 4).

It was confirmed that removal of allergen-specific antibody from currently used histamine-human immunoglobulin complex resulted in disappearance of its suppression effect on the allergen-specific IgE antibody response in animal model of allergy as shown in the table 3 (no statistically significant difference between group 1 and group 3). And a statistically significant difference in the levels of OVA-specific IgE antibody between treatment group 3 (treated by histamine/human immunoglobulin complex incubated with OVA-coupled agarose beads) and treatment group 4 (treated by histamine/human immunoglobulin complex incubated with HSA-coupled agarose beads) (table 3, Student's t-test, p=0.003) firstly demonstrates that allergen-specific antibody contained in the histamine/human immunoglobulin complex plays a critically important role for the suppression mechanism of allergen-specific IgE antibody response induced by treatment with histamine/human immunoglobulin complex in the present experimental condition of example II-4 because the only difference between the treatments of group 3 (adsorbed with OVA-coupled beads) and group 4 (adsorbed with HSA-coupled bead) was presence or absence of OVA-specific antibody as shown in example II-3.

There was no significant difference in levels of OVA-specific IgE antibody between a negative control group treated by normal saline only (group 1) and a group treated by human immunoglobulin only (group 5) (table 3, p=0.812). Levels of OVA-specific IgE antibody were significantly decreased in group 6 treated by simultaneous administration of an immediate mixture of histamine and human immunoglobulin just before the administration compared to negative control group 1 treated by normal saline only (table 3, p<0.006). There was a significant difference in levels of OVA-specific IgE antibody between a group treated by human immunoglobulin only (group 5) and a group treated by simultaneous administration of an immediate mixture of histamine and human immunoglobulin (group 6) (p=0.040). The above statistical analyses of the experimental results in this example II-4 (table 3) logically demonstrate that simultaneous presence of both histamine and allergen-specific antibody is necessary for the suppression of allergen-specific IgE antibody response produced by a histamine/human immunoglobulin complex. Accordingly, the above experiment results demonstrate that the histamine act as a kind of immune response stimulator (adjuvant) and maximized the anti-allergic effect produced by minimal dose of allergen-specific antibody contained in the commercially available histamine-human immunoglobulin formulation through a synergistic effect produced by combination of histamine and allergen-specific antibody. And there were no significant differences in titers of allergen-specific antibody among currently available common immunoglobulin formulations because there is no selection process of immunoglobulin according to the titers of specific antibody to certain allergen in the current manufacturing process of immunoglobulin formulation (FIG. 3, FIG. 4). The above experimental results demonstrate that the currently used immunoglobulin formulations alone show very low anti-allergic effect and thereby is not sufficiently effective for the treatment of allergic disease (table 3, group 5) because currently available common immunoglobulin formulations have very low titers of allergen-specific antibody compared to allergen-specific hyperimmune globulin (FIG. 3, FIG. 4).

Accordingly, the present inventors discovered a presence of synergistic combination effect between histamine and allergen-specific antibody through results of experiments in the above example I-3 and example II-4 for the first time. And the present inventors also demonstrated that the above discovery can be applied to create a new pharmaceutical composition for treating allergic diseases comprising a complex of histamine and allergen-specific antibody that is highly effective and highly advanced compared to the currently used histamine-immunoglobulin complex for treating allergic diseases.

The above results of experiments demonstrate that administrating a complex of histamine and human allergen-specific antibody (that is antibody from mammal species other than mouse) to mouse can suppress allergen-specific allergic immune response observed in the mouse. And the above results of experiments also logically demonstrates that not only a pharmaceutical composition comprising a complex of histamine and human allergen-specific antibody but also a pharmaceutical composition comprising a complex of histamine and allergen-specific antibody obtained from mammals other than human can suppress allergen-specific IgE antibody-mediated allergic immune response in human if the above pharmaceutical composition is administered to human.

II-5. Suppressing Antigen-Specific IgG Antibody Response by a Histamine/Human Anti-OVA Antibody Complex in OVA Allergy Mouse Model

Levels of OVA-specific IgG antibody were measured in the same serum samples obtained from mice of group 1, group 2, and group 3 at day 28 in the same experiment and mice of the above example II-4. The analyzed results were shown in the below table 4.

TABLE 4
Suppression of OVA antigen-specific IgG antibody response by a histamine/human
OVA-specific immunoglobulin complex in OVA allergy mouse model

| | | Levels of | | |
|---|---|---|---|---|
| | | OVA-specific IgG | | |
| | | antibody | *p-value when | *p-value when |
| | Numbers | (mean ± S.D. of | compared with | compared with |
| Experiment groups | of mice | absorbance values) | group 1 | group 2 |
| Group 1- | 8 | 0.834 ± 0.220 | | 0.004 |
| Normal saline treated group | | | | |
| Group 2- | 8 | 0.509 ± 0.075 | 0.004 | |
| His/hIg treated group | | | | |
| Group 3- | 8 | 0.642 ± 0.155 | 0.063 | 0.045 |
| OVA-adsorbed His/hIg treated | | | | |
| group | | | | |

S.D. = standard deviation;
*p-value was calculated by comparing mean values between two groups by Student's t-test (independent sample test) and p value < 0.05 was considered as statistically significantly difference.
His = histamine dichloride,
hIg = human immunoglobulin,
OVA = ovualbumin,
HSA = human serum albumin.

There were statistically significant differences in the mean values of OVA-specific IgG levels among the above 3 groups (Table 4, one-way Anova test, p=0.002). Levels of OVA-specific IgG antibody were significantly decreased in the group 2 mice treated by histamine/human immunoglobulin complex compared to the negative control group mice (group 1) treated by normal saline only (Table 4, p<0.05). Especially, this suppression of OVA-specific IgG antibody response by histamine/human immunoglobulin complex treatment (group 2) was significantly decreased in cases treated by histamine-human immunoglobulin complex that had been incubated with OVA-coupled agarose beads for 16 hours at 4° C. to remove OVA-specific antibody (group 3) (table 4, p=0.045) and there was no significant differences in OVA-specific IgG levels between the above group 3 and negative control group (group 1) (table 4, p=0.063). Accordingly, it can be confirmed that a histamine/antigen-specific antibody complex of the present invention shows a higher efficacy to suppress allergen-specific IgG antibody than the histamine/antigen-nonspecific antibody complex.

Therefore, it can be logically confirmed that a pharmaceutical composition of the present invention comprising histamine and allergen-specific antibody can be used for not only treating IgE-antibody mediated allergic diseases but also treating autoallergic diseases (or autoimmune diseases) including thyroditis, systemic lupus erythematosus, rheumatoid arthritis, or pemphigus that is known to be developed by overproduction of specific IgG antibody to certain autoallergen (or autoantigen) because the above pharmaceutical composition of the present invention has a pharmacological effect that can control IgG antibody response to certain antigen.

### EXAMPLE III

Suppression of House Dust Mite-Specific IgE Antibody-Mediated Allergic Reaction by an Administration of a Histamine/House Dust Mite-Specific Immunoglobulin Complex

III-1. Development of House Dust Mite-Allergy Animal Model

According to the previous reports on the development of house dust mite allergy mouse model (Tournoy K G, et al. Clin Exp Allergy 2000;30:79-85, Yu C K, et al. Clin Exp Allergy 1999;29:414-422), 6 weeks-old male C57B1/6 mouse with body weight ranging from 20 gram to 24 gram were used for the experiments. Forty microgram of house dust mite (Dermatophagoides farinae;

abbreviated as Der f in the followings) protein (quantified by Bradford's method) was mixed with adjuvant containing 2 mg of aluminum hydroxide and 2 mg of magnesium hydroxide (Alumn™, Pierce, Rockford, Ill.) and administered into the above mouse by intraperitoneal injection at day 0.

III-2. Removal of House Dust Mite-Specific Antibody from a Formulation of Histamine-Human Immunoglobulin Complex Using House Dust Mite-Coupled Agarose Bead

To remove the house dust mite-specific antibody in a histamine-human immunoglobulin complex, purified house dust mite extract (Der f, Allergopharma, Germany) or commercially available human serum albumin for intravenous administration (HSA; Green cross Co., Korea) were coupled to the cyanogens-bromide activated agarose beads (Sepharose 4B™, Sigma Chemical Co., St. Louis, Mo.) according to the recommendation of manufacturer. A 16 mg of house dust mite protein or 16 mg of HSA (as a negative control protein) were coupled to 5 ml of the above agarose gel. A 4 ml solution containing 10 mg/ml of histamine-human immunoglobulin complex (Histobulin™, Green cross Co., Korea) was mixed with either 4 ml the above 2 kinds of agarose beads and the mixtures were incubated for 4 hours at room temperature and then the supernatants were separated and the concentrations of immunoglobulin protein in the supernatants were quantified. When house dust mite-specific IgG antibody was detected in the above supernatants by ELISA method as the example II-2, house dust mite-specific IgG antibody was not detected in the above supernatant of histamine-immunoglobulin complex adsorbed by house dust mite-coupled agarose bead and thereby removal of house dust mite-specific IgG antibody from the histamine-human immunoglobulin complex was confirmed. However, house dust mite-specific IgG antibody was still detected in the above supernatant of histamine-immunoglobulin complex adsorbed by HSA-coupled agarose bead and the levels of house dust mite-specific IgG antibody in the supernatant of mixture with HSA-coupled agarose bead did not show statistically significant difference compared to the levels of house dust mite-specific IgG antibody in the original solution of commercially available histamine-immunoglobulin complex.

III-3. Demonstrating a Suppression of House Dust Mite Allergy by a Histamine/House Dust Mite-Specific Antibody Complex

A biological effect of histamine/house dust mite-specific immunoglobulin complex of present invention was demonstrated using 16 mice (6 weeks-old male C57B1/6) that were induced house dust mite allergy as described in the above example III-1. These 16 mice were divided into 2 groups (each groups consisted of 8 mice) and subcutaneously injected with either one of the below 2 kinds of different treatments at 4 hours after injection of house dust mite to induce house dust mite allergy at experiment day 0. The biological effects of the two kinds of treatments were evaluated by measuring the levels of house dust mite-specific IgE antibody in the blood sampled from the tail of the above treated mice at day 15 of the experiment.

Mice of group 1 (house dust mite-specific immunoglobulin depleted histamine/human immunoglobulin complex treated group) received a subcutaneous injection of 2.5 mg per mouse of a histamine/human immunoglobulin complex that depleted house dust mite-specific antibody by prior incubation with house dust mite-coupled agarose beads. Mice of group 2 (histamine/human immunoglobulin complex incubated with HSA-coupled agarose beads treated group) received a subcutaneous injection of 2.5 mg per mouse of a histamine/human immunoglobulin complex supernatant obtained by prior incubation with HSA-coupled agarose beads.

TABLE 5
Suppression of house dust mite-specific IgE antibody-mediated allergic
immune reaction by a histamine/human house dust mite-specific immunoglobulin
complex in a house dust mite-allergy mouse model

| | | Levels of house dust | |
|---|---|---|---|
| | | mite-specific IgE | |
| | | antibody | *p-value when |
| | Numbers of | (mean ± S.D. of | compared with group |
| Experiment groups | mice | absorbance values) | 1 |
| Group 1- Der f-adsorbed His/Ig | 8 | 0.962 ± 0.309 | |
| Group 2- HSA -adsorbed His/Ig | 8 | 0.593 ± 0.282 | 0.026 |

S.D. = standard deviation;
*p-value was calculated by comparing mean values between two groups by Student's t-test (independent sample test) and p value < 0.05 was considered as statistically significantly difference.
His = histamine dichloride,
Ig = immunoglobulin,
Der f = house dust mite (Dermatophagoides farinae),
HSA = human serum albumin.

The results of above experiment shown in the table 5 confirmed that the anti-allergic effect of suppressing the allergen-specific IgE antibody by a histamine/allergen-specific antibody complex of the present invention was operating not only in the OVA allergy mouse model of example I and example II but also in the house dust mite-allergy mouse model. Accordingly, the above results of experiment demonstrate that a pharmaceutical composition of the present invention comprising histamine and allergen-specific antibody for treating allergic diseases can suppress allergic immune reaction to not only inhalant allergen represented by house dust mite but also food allergen represented by ovualbumin (OVA) and can suppress allergic immune reaction to certain allergen regardless the kinds of allergens. Accordingly, the present inventors discovered a new principle producing excellent anti-allergic effect by the synergistic interaction between histamine and allergen-specific antibody for the first time and this principle can be applied for the manufacture of a new pharmaceutical composition of the present invention for preventing and treating allergic diseases that is highly effective and highly advanced compared to the currently existing therapeutic drugs

### INDUSTRIAL APPLICATION

If the pharmaceutical composition of the present invention for treating allergic diseases is administered to the patients with allergic diseases, clinical symptoms of the allergic diseases can be effectively improved by anti-allergic effects of the pharmaceutical composition of the present invention. A pharmaceutical composition of the present invention and a method of manufacturing a pharmaceutical composition of the present invention can be used for the manufacture of medicament for treating allergic diseases as an industrial application. Therefore, if a pharmaceutical composition of the present invention, its use for preventing or treating allergic diseases, and a method of preventing or treating allergic diseases by the above composition was applied, the allergic diseases can be significantly improved even in the patients with refractory allergic diseases who could not be sufficiently improved by standard treatment methods.

1. A pharmaceutical composition for preventing or treating allergic diseases comprising histamine and allergen-specific antibody as active ingredients.

2. The pharmaceutical composition of claim 1, wherein the allergen-specific antibody is an immunoglobulin capable of specifically reacting with allergen.

3. The pharmaceutical composition of claim 1, wherein the allergen-specific antibody is an allergen-specific hyperimmune globulin.

4. The pharmaceutical composition of claim 1, wherein the allergen-specific antibody is an allergen-specific IgG antibody.

5. The pharmaceutical composition of claim 1, wherein the preventing or treating allergic diseases is resulted from the suppression of allergen-specific IgE antibody response.

6. The pharmaceutical composition of claim 5, wherein the preventing or treating allergic diseases is additionally resulted from the suppression of allergen-specific IgG antibody response.

7. The pharmaceutical composition of claim 1, wherein the allergen is one or more of allergen(s) selected from a group consisting of egg, ovualbumin, milk, shrimp, crab, wheat, peanut, house dust mite, pollen, animal dander, and fungus.

8. The pharmaceutical composition of claim 1, wherein the allergen is one or more of allergen(s) selected from a group consisting of nuclear antigen protein, double stranded DNA, phospholipid, beta-2 glycoprotein I, Fc fragment of human IgG antibody and type II collagen.

9. The pharmaceutical composition of claim 1, wherein the allergic diseases are atopic dermatitis, allergic rhinitis, allergic conjunctivitis, urticaria or bronchial asthma.

10. The pharmaceutical composition of claim 8, wherein the allergic diseases are systemic lupus erythematosus or rheumatoid arthritis.

11-22. (canceled)

23. A method of preventing or treating allergic diseases which comprises administrating to a mammal a therapeutically effective amount of histamine and allergen-specific antibody.

24. The method of claim 23, wherein the allergen-specific antibody is an immunoglobulin capable of specifically reacting with allergen.

25. The method of claim 23, wherein the allergen-specific antibody is an allergen-specific hyperimmune globulin.

26. The method of claim 23, wherein the allergen-specific antibody is an allergen-specific IgG antibody.

27. The method of claim 23, wherein the preventing or treating allergic diseases is resulted from the suppression of allergen-specific IgE antibody response.

28. The method of claim 23, wherein the preventing or treating allergic diseases is additionally resulted from the suppression of allergen-specific IgG antibody response.

29. The method of claim 23, wherein the allergen is one or more of allergen(s) selected from a group consisting of egg, ovualbumin, milk, shrimp, crab, wheat, peanut, house dust mite, pollen, animal dander and fungus.

30. The method of claim 23, wherein the allergen is one or more of autoallergen(s) selected from a group consisting of nuclear antigen protein, double stranded DNA, phospholipid, beta-2 glycoprotein I, Fc fragment of human IgG antibody and type II collagen.

31. The method of claim 23, wherein the allergic diseases are atopic dermatitis, allergic rhinitis, allergic conjunctivitis, urticaria or bronchial asthma.

32. The method of claim 30, wherein the allergic diseases are systemic lupus erythematosus or rheumatoid arthritis.

33. The method of claim 25, wherein the allergen-specific hyperimmune globulin is an immunoglobulin fraction purified from a mammal blood containing at least 2 fold higher titer of allergen-specific antibody compared to a blood from normal mammal of same species.

34. The method of claim 25, wherein the allergen-specific hyperimmune globulin is an immunoglobulin fraction purified from a blood of a mammal immunized with an allergen.

35. The pharmaceutical composition of claim 3, wherein the allergen-specific hyperimmune globulin is an immunoglobulin fraction purified from a mammal blood containing at least 2 fold higher titer of allergen-specific antibody compared to a blood from normal mammal of same species.

36. The pharmaceutical composition of claim 3, wherein the allergen-specific hyperimmune globulin is an immunoglobulin fraction purified from a blood of a mammal immunized with an allergen.
